(19) United States
(12) Patent Application Publication
(10) Pub. No.: US 2023/0226348 A1
(43) Pub. Date: Jul. 20, 2023

(54) ELECTRICAL STIMULATION LEAD AND METHODS OF USE

(71) Applicant: Medtronic, Inc., Minneapolis, MN (US)

(72) Inventors: Zhongping Yang, Woodbury, MN (US); Dina L. Williams, Andover, MN (US); Thomas A. Anderson, New Hope, MN (US); Teresa A. Whitman, Dayton, MN (US)

(21) Appl. No.: 18/085,971

(22) Filed: Dec. 21, 2022

Related U.S. Application Data
(60) Provisional application No. 63/301,321, filed on Jan. 20, 2022.

(51) Int. Cl. A61N 1/05

(52) U.S. Cl. CPC A61N 1/0565; A61N 1/0573

(57) ABSTRACT

An electrical stimulation lead includes a lead body having a proximal end and a distal end. A connection interface is coupled to proximal end and a tip electrode is coupled to the distal end. The tip electrode is in electrical communication with the connection interface. A suture line having a barbed structure extends from the tip electrodes. In some examples, the electrical stimulation lead includes a flexible helical electrode capable of engaging tissue. In some examples, the suture line is biodegradable. A method for using an electrical stimulation lead. The method includes placing a tip electrode in a first tissue by pulling the tip electrode into place using a suture line that has a barbed the structure. The method further includes applying electrical stimulation therapy and extracting the tip electrode.

### CROSS-REFERENCE TO RELATED APPLICATION

This application claims the benefit of U.S. Provisional Patent Application No. 63/301,321, filed Jan. 20, 2022, which is incorporated herein by reference in its entirety.

### TECHNICAL FIELD

The current technology generally relates to electrical stimulation. More particularly, the present disclosure relates to an electrical stimulation leads and methods of use. Specifically, the present disclosure relates to electrical stimulation leads configured to allow for removal that may not require invasive surgery and may decrease the likelihood of unwanted tissue damage.

### BACKGROUND

Implantable electrical stimulation leads implanted in a subject's tissue may become dislodged, often requires invasive surgery to remove, and the removal procedure may cause unwanted tissue damage. For example, temporary epicardial pacing and monitoring is necessary after many cardiac surgeries. Post-surgery epicardial pacing is achieved by placing epicardial wires or leads in the heart tissue of a patient. Temporary conventional epicardial wires and/or leads may be challenging to remove often requiring invasive surgery and/or may result in unwanted tissue damage during the removal procedure. Additionally, temporary conventional epicardial wires and/or leads might become dislodged or shift within the heart which may result in inaccurate sensing and unreliable cardiac pacing therapy. Therefore, new technologies are needed to improve electrical stimulation leads.

### SUMMARY

The techniques of this disclosure generally relate to electrical stimulation leads and methods of use.

This disclosure describes, in one aspect, an electrical stimulation lead. The electrical stimulation lead includes a lead body having a proximal end and a distal end. A connection interface is coupled to the proximal end of the lead body. A tip electrode is coupled to the distal end of the lead body. The tip electrode is in electrical communication with the connection interface. The electrical stimulation lead also includes a suture line extending from the tip electrode. The suture line has a barbed structure configured to engage a tissue in which it is placed.

In some embodiments, the suture line is a biodegradable suture line that includes glycolide, dioxanone, trimethylene carbonate, glycolic acid, polymers thereof, copolymers thereof, or combinations thereof. In some embodiments the suture line is configured to degrade within five to 180 days.

In some embodiments the electrical stimulation lead further includes an additional electrode. The additional electrode is coupled to the lead body and is in electrical communication with the connection interface. In some embodiments, the additional electrode is a ring electrode. In some embodiments, the additional electrode is a flexible helical electrode capable of engaging tissue. The flexible helical electrode is disposed towards the distal end of the lead body. In some embodiments, the flexible helical electrode has a first axial length. The first axial length is capable of being elastically expandable to a second axial length. In some embodiments, the flexible helical electrode includes stainless steel, platinum and iridium alloys, nickel, nickel and cobalt alloys, titanium, cobalt, chromium, and molybdenum, or combinations thereof. In some embodiments, the flexible helical electrode has a flare defined by a first diameter at a proximal end of the flexible helical electrode and a second diameter at a distal of the flexible helical electrode. The second diameter is larger than the first diameter.

In some embodiments, the electrical stimulation lead further includes one to six auxiliary electrodes in addition to the tip electrode and the additional electrode. Each auxiliary electrode coupled to the lead body and each auxiliary electrode is in electrical communication with the connection interface. In some embodiments, the tip electrode, the additional electrode, and any auxiliary electrodes are each separated by an interelectrode distance of 1 mm to 15 mm.

In some embodiments, the connection interface has a coaxial configuration.

In another aspect, this disclosure describes a method for using an electrical stimulation lead. Generally, the method includes placing a tip electrode by pulling the tip electrode into place using a suture line that is coupled to the tip electrode. The suture line includes a barbed structure configured to engage a tissue in which it is placed. The method includes applying electrical stimulation therapy though the tip electrode for an amount of time and extracting the electrode.

In some embodiments, the method further includes placing an additional electrode in a first tissue or a second tissue. In some embodiments, the method includes rotating a flexible helical electrode to pierce and engage the first tissue and/or the second tissue. In some embodiments, the method further includes pulling the flexible helical electrode resulting in elastic expansion of the flexible helical electrode.

In some embodiments, the method further includes tying the suture line into a knot.

In some embodiments, the suture line is biodegradable. In some embodiments, the amount of time is the amount of time it takes for the suture line to degrade. In some embodiments, suture line is configured to degrade within five days to 180 days.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A depicts a first view of an example electrical stimulation lead consistent with the technology disclosed herein.

FIG. 1B depicts a second view of the example electrical stimulation lead in FIG. 1A consistent with various embodiments of the present disclosure.

FIG. 2 depicts a schematic cross-sectional facing view of an example electrical stimulation lead consistent with embodiments of the present disclosure.

FIG. 3 is a detailed view of a flexible helical electrode consistent with various embodiments of the present disclosure.

FIG. 4 depicts an electrical stimulation lead consistent with embodiments of the present disclosure.

FIG. 5 depicts an example detail view of a suture line consistent with various embodiments of the present disclosure.

FIG. 6 depicts a flow diagram of a method of using an electrical stimulation lead consistent with embodiments of the present disclosure.

### DETAILED DESCRIPTION

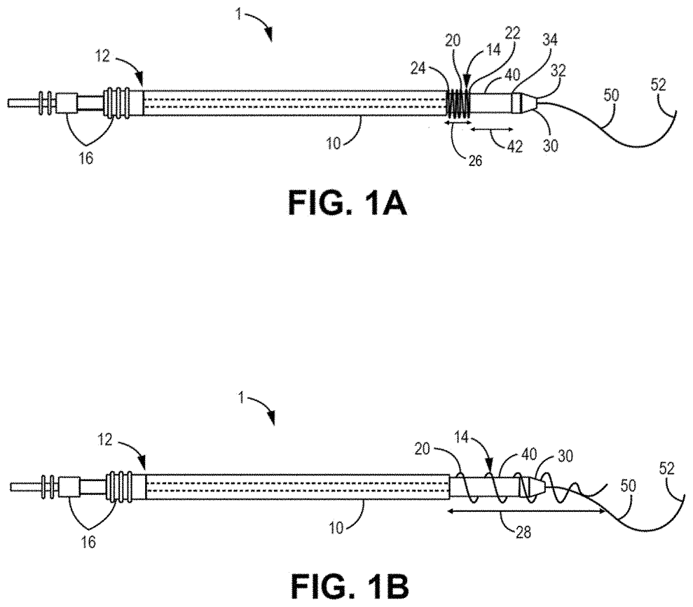

FIG. 1A and FIG. 1B depict a first and second view, respectively, of an example electrical stimulation lead 1 consistent with the present disclosure. The electrical stimulation lead 1 is generally configured to deliver electrical impulses from an electrical stimulation device. In some embodiments, the electrical stimulation lead 1 is an epicardial pacing lead. In some embodiments, the epicardial pacing lead is generally configured to sense cardiac depolarization in the heart tissue of a subject. In some embodiments, the electrical stimulation lead 1 is a neuromuscular electrical stimulation lead. The neuromuscular electrical stimulation lead is generally configured to deliver electrical pulses to produce muscle contractions. For example, neuromuscular electrical stimulation may be used for pain therapy, appetite suppression, and bladder control amongst others. The electrical stimulation lead 1 is generally configured to allow for secure placement in the tissue of a subject. Additionally, in some embodiments, the electrical stimulation lead 1 is generally configured to allow for removal from the patient without requiring invasive surgery. In some embodiments, the electrical stimulation lead 1 is generally configured to reduce the likelihood of imparting unwanted tissue damage during the removal procedure. Furthermore, in some embodiments the electrical stimulation lead 1 is generally configured to allow for temporary ambulatory epicardial pacing.

The electrical stimulation lead 1 generally has a lead body 10. The lead body 10 is generally configured to accommodate electrical communication between an electrical stimulation device and a tip electrode 30. The lead body 10 has a proximal end 12 and a distal end 14. A connection interface 16 is coupled to the lead body 10 at the proximal end 12. The tip electrode 30 is coupled to the distal end 14 of the lead body 10.

In some embodiments, the lead body 10 includes an additional electrode 20. In some embodiments, the additional electrode 20 is a ring electrode. In some embodiments, as depicted in FIG. 1A and FIG. 1B, the additional electrode is a flexible helical electrode, described later herein. In some embodiments, the flexible helical electrode 20 is disposed towards the distal end 14 of the lead body 10. In some embodiments, in addition to the flexible helical electrode 20, the electrical stimulation lead 1 may include one to six auxiliary electrodes (not shown). The auxiliary electrodes may each independently be flexible helical electrode, or a ring electrode as described latter herein.

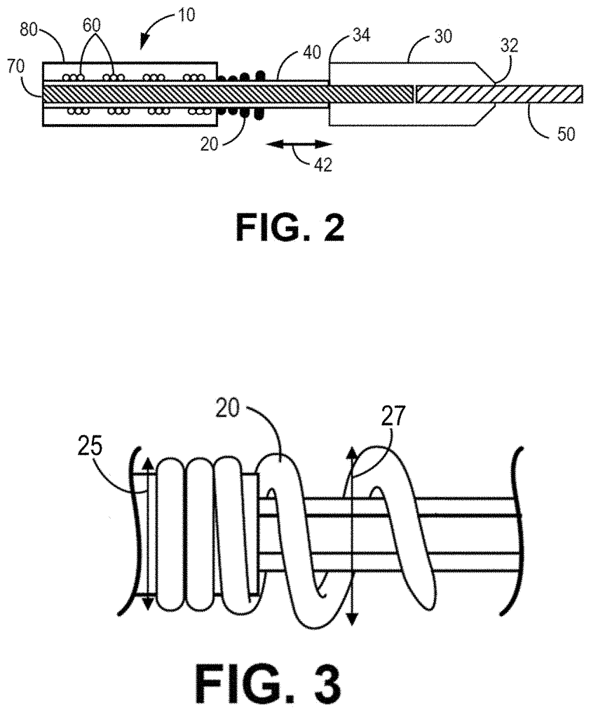

The lead body 10 includes a conductor. As used herein, the term “conductor” refers to any material that is electrically conductive. The conductor is generally configured to provide electrical communication between the tip electrode 30 and the connection interface 16. In some embodiments, where the electrical stimulation lead 1 includes an additional electrode 20, the lead body 10 includes two conductors. Each conductor is generally configured to provide electrical communication between one electrode and the connection interface. In some embodiments where the lead body includes two electrodes, the two conductors may include any pair of suitable electrical conductors, such as coaxial conductors or side-by-side conductors. Examples of side-by-side conductors include “lamp cord” or “zip-cord” conductors known in the art. In an exemplary embodiment shown in FIG. 2, a schematic cross-section of an example lead body 10 consistent with FIGS. lA and 1B is depicted, where the lead body 10 has a pair of coaxial conductors. Specifically, lead body 10 has an inner conductor 70 and an outer conductor 60 helically wound around the inner conductor 70. Helical winding of the outer conductor 60 may impart a high degree of flexibility to lead body 10. In some embodiments, the lead body may include more than two conductors, for example, three conductors, four conductors, five conductors, six conductors, seven conductors, or eight conductors. Generally, the number of conductors is dependent on the number of electrodes.

In some embodiments, each conductor may be independently disposed within an insulative tube. The insulative coatings and/or tubing are generally configured to insulate the conductors from each other and from the external environment outside the lead body 10. In some embodiments, each conductor may be independently coated with an insulative material. In some embodiments, each conductor is independently coated in an insulative material or disposed within an insulative tubing. For example, as depicted in FIG. 2, outer conductor 60 is wound in helical fashion over inner insulative tube 40. In some embodiments, one or more additional inner insulative tubes may be disposed between the inner conductor 70 and the outer conductor 60. In some embodiments the outer conductor may be disposed within an outer insulative tube 80. Examples of insulative tube materials include polyethylene, silastic, neoprene, polypropylene, and polyurethane. Additionally, any insulative tube may be a heat shrink tube. A heat shrink tube is a tube that has a decrease in its internal diameter upon applying heat. Heat shrink tubes may be made of polytetrafloroethelene (PTFE), silicone, polyvinylidene fluoride, polyolefin, or fluorinated ethylene propylene. Insulative coatings include polyoxymethylene UV-cured adhesives, parylene, urethane, poly ether ketone (PEEK), and polyimide.

Returning to FIGS. lA and 1B, the outer surface of lead body 10 may be any biocompatible material. Exemplary biocompatible material include polyurethane; fluoropolymers such as tetrafluroethylene (ETFE), polytetrafluroethylene (PTFE); expanded PTFE such as porous ePTFE and nonporous ePTFE; stainless steel; titanium; titanium alloys; cobalt-chromium alloys; silicone; ceramic; and combinations thereof. In some embodiments, the outer surface of the lead body 10 is defined by the outer insulative tube 80. In some embodiments the outer surface of the lead body 10 is defined by a layer of material disposed outside of the outer insulative tube 80.

The connection interface 16 is generally configured to electrically couple to an electrical stimulation device such as an implantable pacemaker, an external pacing device, or a neuromuscular electrical stimulation device. The connection interface 16 is in electrical communication with the one or more conductors. The connection interface 16 may have a coaxial or side-by-side configuration. An example of a coaxial connector interface configuration is the IS-1 connector.

The connection interface 16 is coupled to the proximal end 12 of the lead body 10. In some embodiments, the connection interface 16 is coupled to the lead body 10 using approaches known in the art. For example, the connection interface 16 can be coupled to lead body 10 using adhesive bonding; crimps or swages; fasteners such as screws, rivets, bolts, and the like; and combinations thereof. In some embodiments, the connection interface 16 can be integral with the lead body 10 such as through welding, soldering, molding, or combinations thereof.

The tip electrode 30 is generally configured to stimulate the tissue of subject. As such, the tip electrode 30 is in electrical communication with the connection interface 16. Through electrical communication with the connection interface 16, the tip electrode 30 is configured to be in electrical communication with the electrical stimulation device. In various embodiments, at least a portion of the tip electrode 30 is generally configured to be positioned in myocardial tissue. In various embodiments, at least a portion of the tip electrode 30 is configured to be positioned in muscle fascia.

At least one conductor is in electrical and mechanical communication with the tip electrode 30 (such as the inner conductor 70 is disposed within the tip electrode 30, as is visible in FIG. 2). The at least one conductor is in electrical and mechanical communication with the tip electrode through coupling techniques known in the art such as mechanical crimp joints, spot welding, fasteners, and the like. In some embodiments, the conductor disposed within the tip electrode 30 may, in some instances, be separate pieces or multiple components that are interconnected (e.g., a multifilar wire). The conductor can also be coated or non-coated cables.

The tip electrode 30 is coupled to the distal end 14 of the lead body 10. The tip electrode 30 is coupled to the lead body 10 through techniques known in the art. The coupling may be achieved via spot welding, crimping, or other suitable mechanism. For example, the tip electrode 30 can be adhesively bonded to lead body 10 and may further include a reduction of (including a portion of) its internal diameter such as via a crimp or swage to increase the attachment force to lead body 10. In an alternative embodiment, tip electrode 30 and the distal end 14 of the lead body 10 may have reverse, mating tapers (e.g., a “Chinese finger grip”) such that an applied tension force causes increased attachment force. A crimp, swage or other geometry modification can also be used to couple the tip electrode 30 to the distal end 14 of the lead body 10.

The tip electrode 30 may be made from any biocompatible conductive material, including platinum, platinum iridium, tantalum, titanium, titanium alloy, conductive polymers, stainless steel, and/or other suitably conductive material.

In some embodiments, the tip electrode 30 is configured to be positioned in the myocardial tissue of an atrium or ventricle by a surgeon during cardiac surgery. Commonly, the tip electrode 30 is place in either the right atrium or the right ventricle. The tip electrode 30 is positioned as to establish electrical communication between the myocardial tissue and the tip electrode 30. Electrical communication between the tip electrode and myocardial tissue is accomplished by physical contact of the tip electrode 30 with the myocardial tissue. The depth at which the tip electrode is positioned within the myocardium may vary according to the patient and application.

In some embodiments, the tip electrode 30 is configured to be positioned in the muscle fascia tissue of a subject. Electrical communication between the tip electrode and muscle fascia tissue is accomplished by physical contact of the tip electrode 30 with the muscle fascia tissue. The depth at which the tip electrode is positioned within the muscle fascia tissue may vary according to the patient and application.

In some embodiments, the tip electrode 30 may have a conical or hemi-spherical distal tip 32 with a relatively narrow tip diameter, e.g., less than 1 mm, for penetrating into and through tissue layers without requiring a sharpened tip or needle-like tip having sharpened or beveled edges that might otherwise produce a cutting action that could lead to lateral displacement of the tip electrode 30 and undesired tissue trauma. In some embodiments, tip electrode 30 may be cylindrical with a relatively flat, blunted or rounded tip. The distal tip of tip electrode 30 may be blunted or rounded to avoid a sharp cutting point or edge. The configuration of the distal tip of the electrode 30 may influence how the surgeon positions the tip electrode 30 in the myocardium.

In some embodiments, the tip electrode 30 may have a maximum diameter at its proximal end 34 where the tip electrode 30 interfaces with lead body 10 with the maximum diameter being isodiametric with lead body 10. The diameter of tip electrode 30 may decrease from the proximal end 34 toward the distal tip 32 of tip electrode 30, e.g., according to a conical or hemispherical shape of the tip electrode 30.

In some embodiments, the lead body 10 includes an additional electrode 20. The additional electrode may be any suitable electrode for use in tissue. In some embodiments, the additional electrode 20 is a ring electrode. In some embodiments, such as shown in FIG. 1A and FIG. 1B, the second electrode 20 is a flexible helical electrode.

In some embodiments, the flexible helical electrode 20 is generally configured to stimulate the tissue of a subject. The flexible helical electrode 20 is disposed toward the distal end 14 of the lead body 10. The flexible helical electrode 20 is disposed toward the proximal end 34 of the tip electrode. The flexible helical electrode has a distal end 22 and a proximal end 24. In various embodiments, the flexible helical electrode 20 is configured to be removed from tissue without requiring invasive surgery. In various embodiments, the flexible helical electrode 20 is configured to decrease the likelihood of imparting unwanted tissue damage during the removal procedure. In various embodiments, the flexible helical electrode 20 is configured to actively engage the tissue thereby preventing dislodgments which might otherwise occur were a straight, non-helical electrode employed.

The coil shape of the flexible helical electrode 20 allows for the active fixation of the flexible helical electrode 20 to tissue. The term “active fixation” refers to fixation of the respective component within tissue at the implant site by intentionally piercing, perforating or penetrating through a tissue surface by the component at the time of implantation. In some embodiments of the present disclosure, active fixation of the flexible helical electrode 20 is achieved by piercing the tissue with the tip of the flexible helical electrode 20 and rotating the lead body 10 such that at least a portion of the flexible helical electrode 20 is screwed into and engages the tissue. Through rotating the flexible helical electrode 20 into the tissue, electrical communication between the flexible helical electrode 20 and the tissue is established by physical contact of the flexible helical electrode 20 with the relevant tissue. In some embodiments, an advantage of the active fixation is the relatively secure placement of the flexible helical electrode 20 within the tissue allowing for the patient to be ambulant.

In some embodiments of the present disclosure, active fixation of the flexible helical electrode 20 is achieved by piercing the epicardium with the tip of the flexible helical electrode 20 and rotating the lead body 10 such that at least a portion of the flexible helical electrode 20 is screwed into and engages myocardial tissue. Additionally, at least a portion of the flexible helical electrode 20 is screwed into and engages epicardial tissue. Through rotating the flexible helical electrode 20 into the heart tissue, electrical communication between the flexible helical electrode 20 and the epicardial and/or myocardial tissues is established by physical contact of the flexible helical electrode 20 with the relevant tissue(s). In some embodiments, an advantage of the active fixation is the relatively secure placement of the flexible helical electrode 20 within the heart tissue allowing for the patient to be ambulant.

In some embodiments of the present disclosure, active fixation of the flexible helical electrode 20 is achieved by piercing the muscle fascia with the tip of the flexible helical electrode 20 and rotating the lead body 10 such that at least a portion of the flexible helical electrode 20 is screwed into and engages muscle fascia. Through rotating the flexible helical electrode 20 into the muscle fascia, electrical communication between the flexible helical electrode 20 and the muscle tissue is established by physical contact of the flexible helical electrode 20 with the muscle fascia. In some embodiments, an advantage of the active fixation is the relatively secure placement of the flexible helical electrode 20 within the muscle fascia allowing for the patient to be ambulant.

The flexible helical electrode 20 is in electrical communication with the connection interface 16. The flexible helical electrode is in electrical communication with at least one conductor allowing for the flexible helical electrode 20 to be in electrical communication with the connection interface 16. In illustrative embodiments, as depicted in FIG. 2, the flexible helical electrode extends from the outer conductor 60 and thus is in electrical communication with outer conductor 60.

In some embodiments, the flexible helical electrode 20 has a flare that is generally configured to enhance the active fixation of the flexible helical electrode 20 to the heart tissue. The flare is defined as the difference between a first diameter 25 at the proximal end 24 of the flexible helical electrode 20 and a second diameter 27 at the distal end 22 of the flexible helical electrode 20 (see FIG. 3).

In some embodiments, the first diameter 25 is greater than 0.5 mm, greater than 1.0 mm, greater than 1.5 mm, greater than 2.0 mm, or greater than 2.5 mm. In some embodiments, the first diameter 25 is less than 3.0 mm, less than 2.5 mm, less than 2.0 mm, less than 1.5 mm, or less than 1.0 mm. In some embodiments, the first diameter 25 is 0.5 mm to 3.0 mm, 0.5 mm to 2.5 mm, 0.5 mm to 2.0 mm, 0.5 mm to 1.5 mm, or 0.5 mm to 1.0 mm. In some embodiments, the first diameter 25 is 1.0 mm to 3.0 mm, 1.0 mm, to 2.5 mm, 1.0 mm to 2.0 mm, or 1.0 mm to 1.5 mm. In some embodiments, the first diameter 25 is 1.5 mm to 3.0 mm, 1.5 mm to 2.5 mm, or 1.5 mm to 2.0 mm. In some embodiments, the first diameter 25 is 2.0 mm to 3.0 mm or 2.5 mm to 2.5 mm. In some embodiments, the first diameter 25 is 2.5 mm to 3.0 mm.

Generally, the second diameter 27 is configured to be greater than the first diameter. In some embodiments, the second diameter 27 is greater than 1.0 mm, greater than 1.5 mm, greater than 2.0 mm, greater than 2.5 mm, greater than 3.0 mm. In some embodiments, the second diameter 27 is less than 3.5 mm, less than 3.0 mm, less than 2.5 mm, less than 2.0 mm, or less than 1.5 mm. In some embodiments, the second diameter 27 is 1.0 mm to 3.5 mm, 1.0 mm to 3.0 mm, 1.0 mm to 2.5 mm, 1.0 mm to 2.0 mm, or 1.0 mm to 1.5 mm. In some embodiments, the second diameter 27 is 1.5 mm to 3.5 mm, 1.5 mm to 3.0 mm, 1.5 mm to 2.5 mm, or 1.5 mm to 2.0 mm. In some embodiments, the second diameter 27 is 2.0 mm to 3.5 mm, 2.0 mm to 3.0 mm, or 2.0 mm to 2.5 mm. In some embodiments, the second diameter 27 is 2.0 mm to 3.5 mm or 2.5 mm to 3.0 mm. In some embodiments, second diameter 27 is 3.0 mm to 3.5 mm.

Given the dimension of the previous paragraphs, in some embodiments, the flare is greater than 0.5 mm, greater than 1.0 mm, greater than 1.5 mm, greater than 2.0 mm, or greater than 2.5. In some embodiments, the flare is less than 3.0 mm, less than 2.5 mm, less than 2.0 mm, less than 1.5 mm, or less than 1.0 mm. In some embodiments, the flare is 0.5 mm to 3.0 mm, 0.5 mm to 2.5 mm, 0.5 mm to 2.0 mm, 0.5 mm to 1.5 mm, or 0.5 mm to 1.0 mm. In some embodiments, the flare is 1.0 mm to 3.0 mm, 1.0 mm to 2.5 mm, 1.0 mm to 2.0 mm, or 1.0 mm to 1.5 mm. In some embodiments, the flare is 1.5 mm to 3.0 mm, 1.5 mm to 2.5 mm, or 1.5 mm to 2.0 mm. In some embodiments, the flare is 2.0 mm to 3.0 mm or 2.0 mm to 2.5 mm. In some embodiments, the flare is 2.5 mm to 3.0 mm.

The flexible helical electrode 20 has a first axial length 26 that is elastically expandable to a second axial length 28 (depicted in FIG. 1A and FIG. 1B, respectively). During a removal procedure, the lead body 10 is pulled, and the flexible helical electrode 20 expands from the first axial length 26 to the second axial length 28. The tissue, which the flexible helical electrode 20 is configured to be engaged to, provides an opposing tensile force, causing the flexible helical electrode 20 to generally straighten during the removal procedure. Generally, the first axial length 26 is shorter than the second axial length 28.

In some embodiments, the first axial length 26 is at least 0.5 mm, at least 1.0 mm, at least 1.5 mm, at least 2.0 mm, at least 2.5 mm, at least 3.0 mm, or at least 3.5 mm. In some embodiments, the first axial length 26 is no greater than 4.0 mm, no greater than 3.5 mm, no greater than 3.0 mm, no greater than 2.5 mm, no greater than 2.0 mm, no greater than 1.5 mm, or no greater than 1.0 mm. In some embodiments, the first axial length 26 is 0.5 mm to 4.0 mm, 0.5 mm to 3.5 mm, 0.5 mm to 3.0 mm, 0.5 mm to 2.5, 0.5 mm to 2.0 mm, 0.5 mm to 1.5 mm, or 0.5 mm to 1.0 mm. In some embodiments, the first axial length 26 is 1.0 mm to 4.0 mm, 1.0 mm to 3.5 mm, 1.0 mm to 3.0 mm, 1.0 mm to 2.5, 1.0 mm to 2.0 mm, or 1.0 mm to 1.5 mm. In some embodiments, the first axial length 26 is 1.5 mm to 4.0 mm, 1.5 mm to 3.5 mm, 1.5 mm to 3.0 mm, 1.0 mm to 2.5, or 1.5 mm to 2.0 mm. In some embodiments, the first axial length 26 is 2.0 mm to 4.0 mm, 2.0 mm to 3.5 mm, 2.0 mm to 3.0 mm, or 2.0 mm to 2.5. In some embodiments, the first axial length 26 is 2.5 mm to 4.0 mm, 2.5 mm to 3.5 mm, or 2.5 mm to 3.0 mm. In some embodiments, the first axial length 26 is 3.0 mm to 4.0 mm or 3.0 mm to 3.5 mm. In some embodiments, the first axial length 26 is 3.5 mm to 4.0 mm.

In some embodiments, the second axial length 28 is at least 2.0 mm, at least 3.0 mm, at least 6.0 mm, at least 9.0 mm, at least 12.0 mm, or at least 15.0 mm. In some embodiments, the second axial length 28 is no greater than 18.0 mm, no greater than 15.0 mm, no greater than 12.0 mm, no greater than 9.0 mm, no greater than 6.0 mm, no greater than 3.0 mm, or no greater than 2.0 mm. In some embodiments, the second axial length 28 is 2.0 mm to 18.0 mm, 2.0 mm to 15 mm, 2.0 mm to 12 mm, 2.0 mm to 9.0 mm, 2.0 mm to 6.0 mm, or 2.0 mm to 3.0 mm. In some embodiments, the second axial length 28 is 3.0 mm to 18.0 mm, 3.0 mm to 15 mm, 3.0 mm to 12 mm, 3.0 mm to 9.0 mm, or 3.0 mm to 6.0 mm. In some embodiments, the second axial length 28 is 6.0 mm to 18.0 mm, 6.0 mm to 15 mm, 6.0 mm to 12 mm, or 6.0 mm. In some embodiments, the second axial length 28 is 9.0 mm to 18.0 mm, 9.0 mm to 15 mm, or 9.0 mm to 12 mm. In some embodiments, the second axial length 28 is 12.0 mm to 18.0 mm or 12.0 mm to 15 mm. In some embodiments, the second axial length 28 is 15.0 mm to 18.0 mm.

The flexible helical electrode 20 may be made of any biocompatible material that is electrically conductive and malleable. Examples of biocompatible materials that are electrically conductive and malleable include platinum-iridium alloys where the iridium content is 10 weight percent or less of the alloy, nickel, nickel-titanium alloy such as nitinol, nickel-cobalt alloys such as MP35N® (SPS Technologies, Inc. based in Jenkintown, PA) titanium, cobalt, chromium, molybdenum, and combinations thereof.

In some embodiments, the tip electrode 30 and the second electrode 20 are separated by an interelectrode distance 42, which is defined as the axial distance between the tip electrode 30 and the second electrode 20. As shown in FIG. 2, in some embodiments, the interelectrode distance 42 is the axial distance between the tip electrode 30 and the flexible helical electrode 20. In some embodiments where the lead body includes auxiliary electrodes, there is an interelectrode distance between each electrode. The interelectrode distance is generally configured to provide the proper spacing of the electrodes for electrical stimulation. In some embodiments, the interelectrode distance is configured to provide the proper spacing for bipolar stimulation. For example, in some embodiments, the interelectrode distance is configured to provide the proper spacing of the electrodes for epicardial pacing. In some embodiments where the lead body includes three or more electrodes, the interelectrode distances are configured to provide the proper spacing for multipolar stimulation. The interelectrode distance may be optimized for anatomy of the subject and/or the electrical stimulation application. In some embodiments, the interelectrode distance is greater than 2 mm, greater than 3 mm, greater than 4 mm, greater than 5 mm, or greater than 10 mm. In some embodiments, the interelectrode distance is less than 15 mm, less than 10 mm, less than 5 mm, less than 4 mm, or less than 3 mm. In some embodiments, the interelectrode distance is 2 mm to 15 mm, 2 mm to 10 mm, 2 mm to 5 mm, 2 mm to 4 mm or 2 mm to 3 mm. In some embodiments, the interelectrode distance is 3 mm to 15 mm, 3 mm to 10 mm, 3 mm to 5 mm, or 3 mm to 4 mm. In some embodiments, the interelectrode distance is 4 mm to 15 mm, 4 mm to 10 mm, or 4 mm to 5 mm. In some embodiments, the interelectrode distance is 5 mm to 15 mm or 5 mm to 10 mm. In some embodiments, the interelectrode distance is 10 mm to 15 mm. Preferably, the interelectrode distance is less than 5 mm.

A suture line 50 extends from the tip electrode 30. The suture line 50 is generally configured to anchor the tip electrode 30 in the tissue. For example, during the implantation procedure of the electrical stimulation lead 1, the suture line 50 may be used to tie one or more sutures that mechanically couple the tip electrode 30 to the tissue. Additionally, in some embodiments, the suture line 50 is generally configured to guide implantation of the electrical stimulation lead 1. For example, during the implantation procedure of electrical stimulation lead 1, the distal end 52 of the suture line 50 may be used to puncture the tissue at the desired tissue location for the tip electrode 30. The suture line can then be pulled such that the tip electrode 30 is placed at the location of the suture line puncture. In some embodiments, to aid in tissue puncture, the distal end 52 of the suture line may include a needle. In some embodiments, an advantage of the suture line 50 is secure placement of the tip electrode within the tissue allowing for the patient to be ambulant during the electrical stimulation therapy. In some embodiments when the flexible helical electrode 20 and the suture line 50 are used in together, an advantage is the secure placement of the electrical stimulation lead 1 allowing for patient ambulation.

In some embodiments, the suture line 50 is biodegradable. As used herein, the term “biodegradable” includes both bioabsorbable and bioresorbable materials. By biodegradable, it is meant that the materials decompose or lose structural integrity under body conditions (e.g., enzymatic degradation, hydrolysis), or are broken down (physically or chemically) under physiologic conditions in the body (e.g., dissolution) such that the degradation products are excretable or absorbable by the body. As used herein “degrade” refers to the amount of time it takes the biodegradable suture line to lose structural integrity. The degradation rate may be affected by the specific location of the suture line 50 within the body.

In the present disclosure, the biodegradable suture line degrades within 5 days to 180 days after affixation in tissue. In some embodiments, the biodegradable suture line degrades within 50 days to 180 days after affixation in tissue. In some embodiments, the biodegradable suture line degrades within 50 days to 100 days after affixation in tissue. In some embodiments, the biodegradable suture line degrades within 50 days to 90 days after affixation in tissue. A biodegradable suture line may be chosen such as to degrade within the time span that the subject is estimated to need electrical stimulation therapy. An advantage of employing a biodegradable suture line is the ability to remove the electrical stimulation lead 1 without having to remove the suture line.

A biodegradable suture line may be made of synthetic materials or natural materials. Suitable synthetic biodegradable materials include polymers such as those made from aliphatic polyesters; polyamides; polyamines; polyalkylene oxalates; poly(anhydrides); polyamidoesters; copoly(ether-esters); poly(carbonates) including tyrosine derived carbonates; poly(hydroxyalkanoates) such as poly(hydroxybutyric acid), poly (hydroxyvaleric acid), and poly(hydroxybutyrate); polyimide carbonates; poly(iminocarbonates) such as poly (bisphenol A-iminocarbonate and the like); polyorthoesters; polyoxaesters including those containing amine groups; polyphosphazenes; poly(propylene fumarates); polyurethanes; polymer drugs such as polydiflunisol, polyaspirin, and protein therapeutics; biologically modified (e.g., protein, peptide) biodegradable polymers; and copolymers, block copolymers, homopolymers, blends, and combinations thereof. Suitable natural biodegradable materials include poly(amino acids) including proteins such as collagen (I, II and III), elastin, fibrin, fibrinogen, silk, and albumin; peptides including sequences for laminin and fibronectin (RGD); polysaccharides such as hyaluronic acid (HA), dextran, alginate, chitin, chitosan, and cellulose; glycosaminoglycan, gut, and combinations thereof. Collagen as used herein includes natural collagen Such as animal derived collagen, gelatinized collagen, or synthetic collagen such as human or bacterial recombinant collagen. In embodiments, glycolide and lactide based polyesters, including copolymers of lactide and glycolide may be used. In some embodiments, the sutures may be coated with a drug, such as an antimicrobial or anti-inflammatory coating. In some embodiments, the biodegradable suture line includes glycolide, dioxanone, trimethylene carbonate, glycolic acid, polymers thereof, copolymers thereof, or combinations thereof.

In some embodiments, the suture line 50 is not biodegradable. Non-biodegradable suture lines may be made of natural or synthetic materials. Suitable non-absorbable natural materials include cotton, silk, and rubber. Suitable non-biodegradable materials include polyolefins such as polyethylene (including ultra-high molecular weight polyethylene) and polypropylene including atactic, isotactic, syndio tactic, and blends thereof; polyethylene glycols; polyethylene oxides; ultra-high molecular weight polyethylene; copolymers of polyethylene and polypropylene; polyisobutylene and ethylene-alpha olefin copolymers; fluorinated polyolefins such as fluoroethylenes, fluoropropylenes, fluoro PEGs, and polytetrafluoroethylene; polyamides such as nylon, Nylon 6, Nylon 6.6, Nylon 6,10, Nylon 11, Nylon 12, and polycaprolactam, polyamines; polyimines; polyesters such as polyethylene terephthalate, polyethylene naphthalate, polytrimethylene terephthalate, and polybutylene terephtha late; polyethers; polytetramethylene ether glycol; polybutesters, including copolymers of butylene terephthalate and poly tetramethylene ether glycol, 1,4-butanediol; polyurethanes; acrylic polymers; methacrylics; vinyl halide polymers and copolymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as poly vinyl acetate; copolymers of vinyl monomers with each other and olefins such as ethylene-methyl methacrylate copolymers; acrylonitrile-styrene copolymers; acrylonitrile, butadiene and styrene (ABS) resins; ethylene-vinyl acetate copolymers; alkyd resins; polycarbonates; polyoxymethylenes; polyphosphazine; polyimides; epoxy resins; aramids; rayon; rayon-triacetate; Spandex; silicones; and copolymers and combinations thereof. Additionally, non-biodegradable polymers and monomers may be combined with each other. Polypropylene can also be utilized to form the suture. The polypropylene can be isotactic polypropylene or a mixture of isotactic and syndiotactic or atactic polypropylene. Additionally, non-absorbable synthetic and natural polymers and monomers may be combined with each other and may also be combined with various absorbable polymers and monomers to create fibers and filaments.

In some embodiments, the suture line 50 has a barbed structure, an example of which is visible in the detail view provided by FIG. 5 (the suture line in FIG. 5 is referenced as element 500). The barbed structure is generally configured to allow for active fixation of the suture line 50 to the tissue. The barbed structure includes a central wire 520 and a plurality of barbs 510. The plurality of barbs 510 are disposed along the surface of the central wire 520. Each barb of the plurality of barbs 510 is configured to pierce and engage the tissue preventing the suture line from shifting and/or dislodging once affixed to the tissue. Each of the plurality of barbs is pointed generally away from the direction that the suture line 50 is inserted into the tissue, facilitating passage in one direction through the tissue (e.g., for insertion into the tissue), and discouraging passage in the opposite direction (e.g., for removal from the tissue) suture line. The barbed structure may advantageously reduce reliance on suture knots. Sutures typically employ a knot at the distal end to secure the suture line end in the tissue. Knot tying adds time to a procedure and may result in additional bulk material being left at the wound site. In some embodiments, however, a knot is tied when a suture line having a barbed structure is employed.

Each barb in the plurality of barbs 510 has an angle 530 and a length 540. The angle 530A is the angle at with the barb protrudes from the central wire 520. The length 540 is the distance the barb protrudes from the central wire 520. The length 540 and angle 530A are generally designed so that the barb engages the tissue. The angle 530A and length 540 can be tuned to achieve a desired strength of active fixation. In some embodiments, each barb in the plurality of barbs 510 can have a different angle 530A. In some embodiments, each barb in the plurality of barbs 510 has the same angle 530A. In some embodiments, a single barb may include more than one angle. The plurality of barbs 510 can be arranged in any suitable pattern along the central wire 520 including in a helical, linear, or randomly spaced pattern with respect to longitudinal axis of the central wire 520. The surface area of each barb of the plurality of barbs 510 can vary. For example, barbs with a larger surface area, barbs with a smaller surface area, or a combination barb with different surface areas may be used. In some embodiments, the central wire 520 may include a staggered arrangement of barbs with a relatively long length 540 or barbs with a relatively short length 540. In other embodiments, a random configuration barbs with a relatively long length 540 or barbs with a relatively short length 540 may be used. The pattern of the plurality of barbs 510 may be symmetrical or asymmetrical.

A suture line having a barbed structure can be made of a biodegradable material or non- biodegradable material. Examples of biodegradable materials and non- biodegradable materials were described previously. Commercially available examples of suture lines that are not biodegradable and have a barbed structure include V-LOC™ PBT (Medtronic Inc., based in Fridley, Minn., USA). Commercially available examples of suture lines that are biodegradable and have a barbed structure include V-LOC™ 90 (Medtronic Inc.) and V-LOC™ 180 (Medtronic Inc.). In various examples consistent with the technology disclosed herein, the suture line lacks a barbed structure.

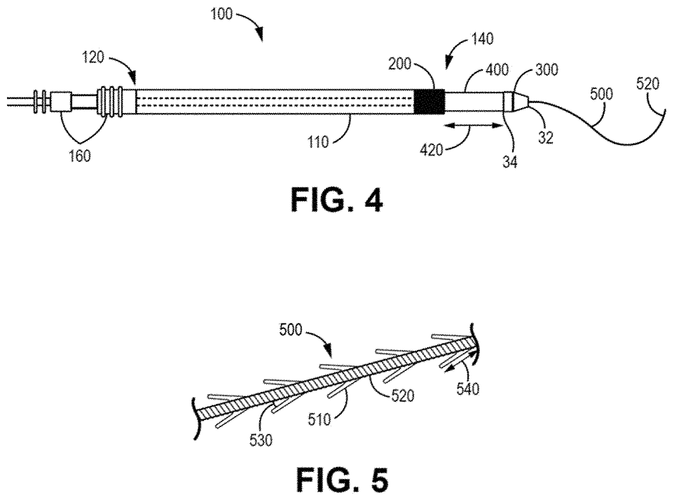

Consistent with another embodiment of the present disclosure, FIG. 4 depicts a view of an electrical stimulation lead 100. The electrical stimulation lead 100 is generally configured to deliver electrical impulses from an electrical stimulation device. In some embodiments, the electrical stimulation lead 100 is an epicardial pacing lead. In some embodiments, the epicardial pacing lead is generally configured to sense cardiac depolarization in the heart tissue of a subject. In some embodiments, the electrical stimulation lead 100 is a neuromuscular electrical stimulation lead. The neuromuscular electrical stimulation lead is generally configured to deliver electrical pulses to produce muscle contractions. The electrical stimulation lead 100 is generally configured to allow for secure placement in the tissue of a subject. Additionally, in some embodiments, the electrical stimulation lead 100 is generally configured to allow for removal from the patient without requiring invasive surgery. In some embodiments, the electrical stimulation lead 100 is generally configured to reduce the likelihood of imparting unwanted tissue damage during the removal procedure. Furthermore, in some embodiments the electrical stimulation lead 100 is generally configured to allow for temporary ambulatory epicardial pacing.

The electrical stimulation lead 100 generally has a lead body 110. The lead body 110 is generally configured accommodate electrical communication between an electrical stimulation device and a tip electrode 300. The lead body 110 has a proximal end 120 and a distal end 140. A connection interface 160 is coupled to the lead body 110 at the proximal end 120. The tip electrode 300 is coupled to the distal end 140 of the lead body 110. A suture line 500 extends from the tip electrode 300.

The lead body 110 is generally configured facilitate electrical communication between an electrical stimulation device and the tip electrode 300. The lead body 110 may be any configuration or material discussed elsewhere herein.

The connection interface 160 is generally configured to electrically couple to an electrical stimulation device such as an implantable pacemaker, an external pacemaker, or a neuromuscular electrical stimulation device. The connection interface is coupled to the proximal end 120 of the lead body 110. The connection interface 160 may have any configuration discussed elsewhere herein.

The tip electrode 300 is generally configured to stimulate the tissue of subject. As such, the tip electrode 300 is configured to be in electrical communication with the connection interface 160. Through electrical communication with the connection interface 160, the tip electrode 300 is configured to be in electrical communication with the electrical stimulation device. At least a portion of the tip electrode 300 is generally configured to be implantable in the tissue of a subject. The tip electrode 300 may have any configuration and be made of any material discussed elsewhere herein. Additionally, the tip electrode 300 may be positioned within the tissue(s) in a manner as discussed elsewhere herein.

In some embodiments, a second electrode 200 disposed towards the distal end 140 of the lead body 110. The second electrode 200 is generally configured to stimulate the tissue of subject. In some embodiments, the second electrode 200 is generally configured such that at least a portion of the second electrode is within the myocardium tissue and/or at least portion of the second electrode is within the epicardial tissue.

The second electrode 200 is generally configured to be in electrical communication with the connection interface 160. The second electrode 200 is in electrical communication with at least one conductor of the lead body 110 allowing for the second electrode 200 to be in electrical communication with the connection interface 160.

In the current example, the second electrode 200 is not a flexible helical electrode. In some embodiments, the second electrode may be a single continuous ring electrode. In some such examples, portions of the ring may be coated with an electrically insulating coating, e.g., parylene, polyurethane, silicone, epoxy, or other insulating coating, to reduce the electrically conductive surface area of the ring electrode. For instance, one or more sectors of the ring may be coated to separate two or more electrically conductive exposed surface areas of the second electrode. In some embodiments, the ring electrode substantially encircles the longitudinal axis of the lead body.

The second electrode 200 is placed within the tissue in order establish electrical communication between the tissue and the second electrode 200. Electrical communication between the second electrode 200 and tissue is accomplished by physical contact of the second electrode 200 with the tissue.

The second electrode 200 may be made from any biocompatible conductive material, including platinum, platinum iridium, tantalum, titanium, titanium alloy, conductive polymers, and/or other suitably conductive material. In some embodiments, the lead body 110 may include auxiliary electrodes, for example, three electrodes, four electrodes, five electrodes, six electrodes, seven electrodes, or eight electrodes. The auxiliary electrodes may each independently be any suitable electrode for use within the body, examples are discussed elsewhere herein.

The tip electrode 300 and the second electrode 200 are separated by an interelectrode distance 420, the interelectrode distance 420 generally configured to provide suitable spacing of the electrodes for applying electrical stimulation therapy. In some embodiments where the lead body 110 includes more than two electrodes, there is an interelectrode distance between each electrode. The interelectrode distance 420 may be any interelectrode distance discussed elsewhere in herein.

A suture line 500 extends from the tip electrode 300. The suture line 500 is generally configured to anchor the tip electrode 300 in the tissue. In some embodiments, the suture line 500 has a barbed structure. The barbed structure of the suture line 500 is generally configured to pierce and engage the tissue in which it is placed. The suture line 500 materials and properties were discussed elsewhere herein. In some embodiments, the suture line 500 is biodegradable. Suture lines that are biodegradable are discussed elsewhere herein. Suture lines that have a barbed structure are discussed elsewhere herein. Suture lines that are biodegradable and have a barbed structure are discussed elsewhere herein.

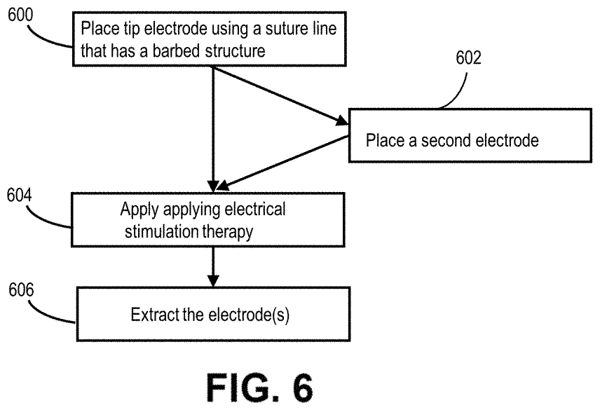

FIG. 6 describes a method for using an electrical stimulation lead consistent with the present disclosure. Generally, the method includes the steps of placing the tip electrode into the tissue 600, applying electrical stimulation therapy 604, and extracting the tip electrode form the tissue 606. In some embodiments, the electrical stimulation lead is provided as previously describe relative to FIG. 1A, FIG. 1B, FIG. 2, FIG. 3, and FIG. 5.

Placing the tip electrode into the tissue 600 includes establishing electrical communication between the tissue and the tip electrode. Electrical communication between the tip electrode and the 1 tissue is accomplished by physical contact of the tip electrode with the tissue. The depth at which the tip electrode is placed in the tissue may vary according to the patient and application. In some embodiments, the tip electrode is placed into heart tissue such as the myocardium and/or the epicardium. In some embodiments, the tip electrode is placed in the muscle fascia.

Placing the tip electrode into the tissue includes pulling the tip electrode into place using a suture line that has a barbed structure. The suture line is coupled to the tip electrode. The suture line is generally configured to guide implantation of the electrical stimulation lead 1. For example, during the implantation procedure of electrical stimulation lead suture line may be used to puncture the tissue at the desired tissue location for the tip electrode. The suture line can then be pulled such that the tip electrode is placed at the location of the suture line puncture. In some embodiments, securing the tip electrode in the desired location provides a potential advantage of preventing the movement of the tip electrode during the other steps of the implantation procedure. In some embodiments, the suture line can be used to secure the tip electrode preventing movement of the tip electrode from its desired placement during electrical stimulation treatment. In some embodiments, the suture line may be tied into a knot. The knot may enhance the ability of the suture wire to hold the tip electrode in place. The barbed structure of the suture line is generally configured to allow for active fixation of the suture line 50 to the tissue. Each barb of the plurality of barbs 510 is configured to pierce and engage the tissue preventing the suture line from shifting and/or dislodging once affixed to the tissue (FIG. 5).

In some embodiments when the electrical stimulation device is an epicardial stimulation device, the tip electrode is generally placed 600 during cardiac surgery. The tip electrode may be placed 600 in the myocardial tissue of an atrium or a ventricle. Commonly for epicardial pacing, the tip electrode is placed in either the right atria or the right ventricle.

In some embodiments, the suture line may be biodegradable. In some embodiments, the suture line is not biodegradable. In some embodiments, the suture line has a barbed structure. In some embodiments, the suture line is not biodegradable and has a barbed structure. In some embodiments, the suture line is biodegradable and has a barbed structure. The materials, properties, and structures of applicable suture lines are described elsewhere herein.

In some embodiments, a second electrode is placed in the tissue 602. In some embodiments, the second electrode is a ring electrode. In some embodiments, the second electrode is a flexible helical electrode. Placing the flexible helical electrode includes rotating a flexible helical electrode which allows the flexible helical electrode to pierce and engage the tissue through active fixation. When the flexible helical electrode has a right-hand screw configuration, the flexible helical electrode is rotated clockwise. When the flexible helical electrode has a left-hand screw configuration, the flexible helical electrode is rotated counterclockwise. The number of full rotations and partial rotations depends on the application. Generally, the flexible helical electrode is rotated until at least a portion of the flexible helical electrode pierces and engages one or more tissues.

In some embodiments when the electrical stimulation lead is an epicardial pacing lead, rotating the flexible helical electrode allows the flexible helical electrode to pierce and engage the heart tissue through active fixation. Upon reaching the epicardium, the electrical stimulation lead is rotated to screw the flexible helical electrode into the heart tissue. In some embodiments, the flexible helical electrode is rotated until at least a portion of the flexible helical electrode pierces and engages myocardial tissue and at least a portion of the flexible helical electrode pierces and engages the epicardial tissue.

Electrical stimulation therapy is applied 604 through the tip electrode for an amount of time. In some embodiments, the electrical stimulation therapy is ambulatory electrical stimulation therapy. In some embodiments, the electrical stimulation therapy is temporary. In some embodiments, the electrical stimulation therapy may be for at least 5 days, at least 20 days, at least 50 days, or at least 90 days. In some embodiments, the electrical stimulation therapy may be for less than 180 days, less than 90 days, less than 50 days, or less than 20 days. In some embodiments, electrical stimulation therapy may be applied for 5 days to 20 days, 5 days to 50 days, 5 to 90 days, or 5 to 180 days. In some embodiments, the electrical stimulation therapy may be applied for 20 days to 50 days, 20 to 90 days, or 20 to 180 days. In some embodiments, the electrical stimulation therapy may be applied for 50 days to 90 days or 50 days to 180 days. In some embodiments, the electrical stimulation therapy may be applied for 90 days to 180 days. In some embodiments where a biodegradable suture line is employed, the electrical stimulation therapy may be applied for the duration in which the biodegradable suture line is not degraded.

The tip electrode is extracted 606 from the tissue. To extract the tip electrode, the electrical stimulation lead is pulled from the body. In some embodiments where a biodegradable suture line is employed, the removal of the electrode is after the degradation of the suture line. For example, if the suture line degrades in 10 days, then the electrode may be removed on the tenth day or later.

In some embodiments where the electrical stimulation lead includes a second electrode, the tip electrode and the second electrode are extracted from the tissue 606. To extract the tip electrode and the second electrode, the electrical stimulation lead is pulled from the body. In some embodiments where a biodegradable suture line is employed, the removal of the electrode is after the degradation of the suture line. For example, if the suture line degrades in 10 days, then the electrode may be removed on the tenth day or later.

In some embodiments where the electrical stimulation lead includes a flexible helical electrode, both the flexible helical electrode and the tip electrode are extracted 606 from the tissue. To extract the flexible helical electrode, a pulling force is applied to the flexible helical electrode resulting in elastic expansion of the flexible helical electrode. The tissue in which the flexible helical electrode is configured to be implanted provides resistance to the pulling force, resulting in straightening of the flexible helical electrode to facilitate removal. As a result of pulling the flexible helical electrode, the flexible helical electrode is extracted from the tissue and the tip electrode is extracted from the tissue.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. An electrical stimulation lead comprising:
a lead body having a proximal end and a distal end;
a connection interface coupled to the proximal end;
a tip electrode coupled to the distal end, the tip electrode in electrical communication with the connection interface; and
a suture line extending from the tip electrode, the suture line having a barbed structure configured to engage a tissue in which it is placed.

2. The electrical stimulation lead of claim 1, wherein the suture line is a biodegradable suture line comprises glycolide, dioxanone, trimethylene carbonate, glycolic acid, polymers thereof, copolymers thereof, or combinations thereof.

3. The electrical stimulation lead of claim 2, wherein the suture line is configured to degrade within five days to 180 days.

4. The electrical stimulation lead of claim 1, further comprising an additional electrode coupled to the lead body, wherein the additional electrode is in electrical communication with the connection interface.

5. The electrical stimulation lead of claim 4, wherein the additional electrode is a ring electrode.

6. The electrical stimulation lead of claim 4, wherein the additional electrode is a flexible helical electrode capable of engaging tissue, the flexible helical electrode disposed towards the distal end, the flexible helical electrode having a first axial length and being elastically expandable to a second axial length.

7. The electrical stimulation lead of claim 6, wherein the flexible helical electrode comprises stainless steel, platinum and iridium alloys, nickel, nickel and cobalt alloys, titanium, cobalt, chromium, and molybdenum, or combinations thereof.

8. The electrical stimulation lead of claim 6, wherein the flexible helical electrode has a flare defined by a first diameter at a proximal end of the flexible helical electrode and a second diameter at a distal of the flexible helical electrode, wherein the second diameter is larger than the first diameter.

9. The electrical stimulation lead of claim 4, further comprising one to six auxiliary electrodes, each coupled to the lead body and each in electrical communication with the connection interface.

10. The electrical stimulation lead of claim 9, wherein each of the auxiliary electrodes is a ring electrode or a flexible helical electrode.

11. The electrical stimulation lead of claim 9, wherein the tip electrode, the additional electrode, and any auxiliary electrodes are each separated by an interelectrode distance, the interelectrode distance being 1 mm to 15 mm.

12. The electrical stimulation lead of claim 4, wherein the connection interface has a coaxial configuration.

13. A method for using an electrical stimulation lead, the method comprising:
placing a tip electrode in a first tissue by pulling the tip electrode into place using a suture line that is coupled to the tip electrode, the suture line having a barbed structure configured to engage a tissue in which it is placed;
applying electrical stimulation therapy through the tip electrode for an amount of time; and extracting the tip electrode.

14. The method of claim 13, further comprising placing an additional electrode in the first tissue or a second tissue.

15. The method of claim 14, further comprising rotating a flexible helical electrode to pierce and engage the first tissue and/or the second tissue.

16. The method of claim 15, further comprising pulling the flexible helical electrode resulting in elastic expansion of the flexible helical electrode.

17. The method of claim 14, wherein the method further comprises tying the suture line into a knot.

18. The method of claim 13, wherein the suture line is biodegradable.

19. The method of claim 18, wherein the amount of time is the amount of time it takes for the suture line to degrade.

20. The method of claim 18, wherein the suture line is configured to degrade within five days to 180 days.
